(19) United States
(12) Patent Application Publication
(10) Pub. No.: US 2024/0395403 A1
(43) Pub. Date: Nov. 28, 2024

(54) Post-Acute Care System and Method

(71) Applicant: RevelAi Inc., Durham, NC (US)

(72) Inventors: Christian Pean, Durham, NC (US); Hadi Javeed, Saugus, MA (US)

(21) Appl. No.: 18/674,015

(22) Filed: May 24, 2024

Related U.S. Application Data
(60) Provisional application No. 63/503,969, filed on May 24, 2023.

(51) Int. Cl. G16H 50/20; G16H 10/60; G16H 15/00; G16H 40/20; G16H 70/20; G16H 80/00

(52) U.S. Cl. CPC G16H 50/20; G16H 10/60; G16H 15/00; G16H 40/20; G16H 70/20; G16H 80/00

(57) ABSTRACT

A computer-implemented method, computer program product and computing system for: detecting the discharge of a patient from an acute care facility; deploying one or more AI agents to perform one or more tasks associated with a post-acute care transition phase of the patient; and analyzing interactions between the patient and the one or more AI agents to identify one or more urgent issues.

### RELATED APPLICATION(S)

This application claims the benefit of U.S. Provisional Application No. 63/503,969, filed on 24 May 2023, the entire contents of which are herein incorporated by reference.

### TECHNICAL FIELD

This disclosure relates to post-acute care systems and, more particularly, to AI-enhanced post-acute care systems.

### BACKGROUND

Post-acute care has evolved significantly over the past century, originating in the early 20th century with convalescent homes and rehabilitation facilities run by charitable organizations. These early establishments aimed to provide care for patients recovering from illnesses or surgeries. The demand for rehabilitation services saw a significant increase after World War II due to the high number of wounded soldiers, with the Veterans Administration (VA) hospitals playing a crucial role in advancing rehabilitation services. During this period, physical and occupational therapy became more widely recognized and utilized.

The 1960s marked a major milestone with the establishment of the Medicare and Medicaid programs in the United States in 1965, which provided funding for skilled nursing facilities (SNFs), home health agencies (HHAs), and inpatient rehabilitation facilities (TRFs). This led to substantial growth in post-acute care services. The subsequent decades saw a shift towards managed care and cost-containment measures, emphasizing the need to discharge patients from hospitals as soon as they were medically stable, thus increasing the demand for post-acute care services. In the 1990s, the Balanced Budget Act of 1997 introduced the Prospective Payment System (PPS) for SNFs to control costs by setting predetermined rates for care, followed by similar payment reforms for other post-acute care settings.

From the 2000s to the present, there has been a focus on value-based care and integrated care models. The Affordable Care Act (ACA) of 2010 further emphasized reducing hospital readmissions and improving the quality of care in post-acute settings through initiatives like the Hospital Readmissions Reduction Program (HRRP). Various types of post-acute care have emerged, including skilled nursing facilities (SNFs), inpatient rehabilitation facilities (TRFs), home health agencies (HHAs), and long-term acute care hospitals (LTACHs), each catering to specific patient needs.

Despite its evolution, post-acute care faces several shortcomings. Fragmentation and lack of coordination are significant issues, as patients often transition between different providers, leading to gaps in care, medication errors, and poor communication among providers. This lack of continuity can negatively impact patient outcomes and increase the risk of hospital readmissions. Additionally, the quality and consistency of care can vary widely between providers. Some facilities may lack adequate staffing, resources, or specialized expertise, and while regulations exist to ensure quality, enforcement can be inconsistent.

Financial and reimbursement challenges further complicate the landscape of post-acute care. Payment models can be complex and may not adequately cover the costs of high-quality care. Efforts to contain costs sometimes result in under-provision of care or incentivize shorter lengths of stay, which may not always align with patient needs. Access and equity issues also persist, with disparities based on geographic location, socioeconomic status, and insurance coverage. Patients in rural or underserved areas may have limited access to post-acute care services, affecting their recovery and long-term outcomes.

Workforce challenges, including staffing shortages and the need for continuous training and education, pose additional hurdles. Ensuring that staff are adequately trained and up-to-date with best practices is essential but often difficult to achieve. Technological integration is another area of concern. Many post-acute care providers lack advanced health information technology systems, hindering data sharing and coordination of care. While telehealth has the potential to improve access and continuity of care, its adoption in post-acute settings has been slow and uneven.

Addressing these shortcomings requires ongoing efforts to improve care coordination, ensure consistent quality standards, enhance reimbursement models, and expand access to care.

### Summary of Disclosure

In one implementation, a computer-implemented method is executed on a computing device and includes: detecting the discharge of a patient from an acute care facility; deploying one or more AI agents to perform one or more tasks associated with a post-acute care transition phase of the patient; and analyzing interactions between the patient and the one or more AI agents to identify one or more urgent issues.

One or more of the following features may be included. Detecting the discharge of a patient from an acute care facility may include: processing at least a portion of an Admission, Discharge, and Transfer (ADT) feed. The one or more AI agents may include one or more of the following: a transitions of care history interview and discharge review agent; a medication reconciliation agent; a medical knowledge query response agent; a community resources agent; and an appointment scheduling and confirmation agent. In response to identifying the one or more urgent issues, the one or more urgent issues may be escalated to one or more healthcare professionals. Deploying one or more AI agents to perform one or more tasks associated with a post-acute care transition phase of the patient may include: enabling the one or more AI agents to interact with the patient. Interactions between the patient and the one or more AI agents may be summarized to generate an interaction summary. Review of the interaction summary by one or more healthcare professionals may be enabled. The AI agents may be enabled to collaborate with each other to perform the one or more tasks associated with the post-acute care transition phase of the patient. The one or more AI agents may be enabled to collaborate with one or more healthcare professionals to perform the one or more tasks associated with the post-acute care transition phase of the patient. Communications may be enabled with one or more of: a healthcare database, a healthcare platform, and a governmental/information database.

In another implementation, a computer program product resides on a computer readable medium and has a plurality of instructions stored on it. When executed by a processor, the instructions cause the processor to perform operations including: detecting the discharge of a patient from an acute care facility; deploying one or more AI agents to perform one or more tasks associated with a post-acute care transition phase of the patient; and analyzing interactions between the patient and the one or more AI agents to identify one or more urgent issues.

One or more of the following features may be included. Detecting the discharge of a patient from an acute care facility may include: processing at least a portion of an Admission, Discharge, and Transfer (ADT) feed. The one or more AI agents may include one or more of the following: a transitions of care history interview and discharge review agent; a medication reconciliation agent; a medical knowledge query response agent; a community resources agent; and an appointment scheduling and confirmation agent. In response to identifying the one or more urgent issues, the one or more urgent issues may be escalated to one or more healthcare professionals. Deploying one or more AI agents to perform one or more tasks associated with a post-acute care transition phase of the patient may include: enabling the one or more AI agents to interact with the patient. Interactions between the patient and the one or more AI agents may be summarized to generate an interaction summary. Review of the interaction summary by one or more healthcare professionals may be enabled. The AI agents may be enabled to collaborate with each other to perform the one or more tasks associated with the post-acute care transition phase of the patient. The one or more AI agents may be enabled to collaborate with one or more healthcare professionals to perform the one or more tasks associated with the post-acute care transition phase of the patient. Communications may be enabled with one or more of: a healthcare database, a healthcare platform, and a governmental/information database.

In another implementation, a computing system includes a processor and a memory system configured to perform operations including: detecting the discharge of a patient from an acute care facility; deploying one or more AI agents to perform one or more tasks associated with a post-acute care transition phase of the patient; and analyzing interactions between the patient and the one or more AI agents to identify one or more urgent issues.

One or more of the following features may be included. Detecting the discharge of a patient from an acute care facility may include: processing at least a portion of an Admission, Discharge, and Transfer (ADT) feed. The one or more AI agents may include one or more of the following: a transitions of care history interview and discharge review agent; a medication reconciliation agent; a medical knowledge query response agent; a community resources agent; and an appointment scheduling and confirmation agent. In response to identifying the one or more urgent issues, the one or more urgent issues may be escalated to one or more healthcare professionals. Deploying one or more AI agents to perform one or more tasks associated with a post-acute care transition phase of the patient may include: enabling the one or more AI agents to interact with the patient. Interactions between the patient and the one or more AI agents may be summarized to generate an interaction summary. Review of the interaction summary by one or more healthcare professionals may be enabled. The AI agents may be enabled to collaborate with each other to perform the one or more tasks associated with the post-acute care transition phase of the patient. The one or more AI agents may be enabled to collaborate with one or more healthcare professionals to perform the one or more tasks associated with the post-acute care transition phase of the patient. Communications may be enabled with one or more of: a healthcare database, a healthcare platform, and a governmental/information database.

The details of one or more implementations are set forth in the accompanying drawings and the description below. Other features and advantages will become apparent from the description, the drawings, and the claims.

### BRIEF DESCRIPTION OF THE DRAWINGS

FIG. 1 is a diagrammatic view of a post-acute care transition process in accordance with various embodiments of the present disclosure;

FIG. 2 is a flow chart of one implementation of the post-acute care transition process of FIG. 1 in accordance with various embodiments of the present disclosure; and

FIG. 3 is a diagrammatic view of a computer system and the post-acute care transition process of FIG. 1 coupled to a distributed computing network in accordance with various embodiments of the present disclosure.

Like reference symbols in the various drawings indicate like elements.

### DETAILED DESCRIPTION OF THE PREFERRED EMBODIMENTS

As will be discussed in greater detail below, implementations of the present disclosure may detect the discharge of a patient from an acute care facility and deploy AI agents to perform tasks associated with a post-acute care transition phase of the patient. Interactions between the patient and the one or more AI agents may be analysed to identify one or more urgent issues.

The details of one or more implementations are set forth in the accompanying drawings and the description below. Other features and advantages will become apparent from the description, the drawings, and the claims.

### Post-Acute Care Transition Process:

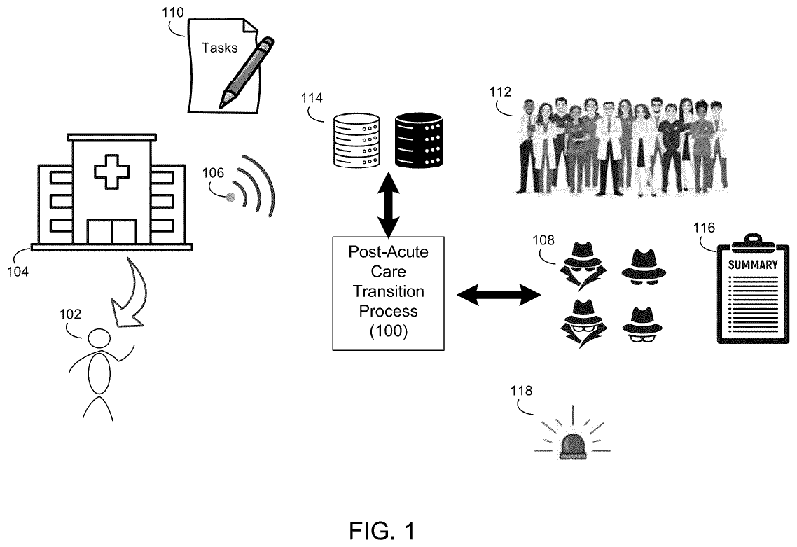

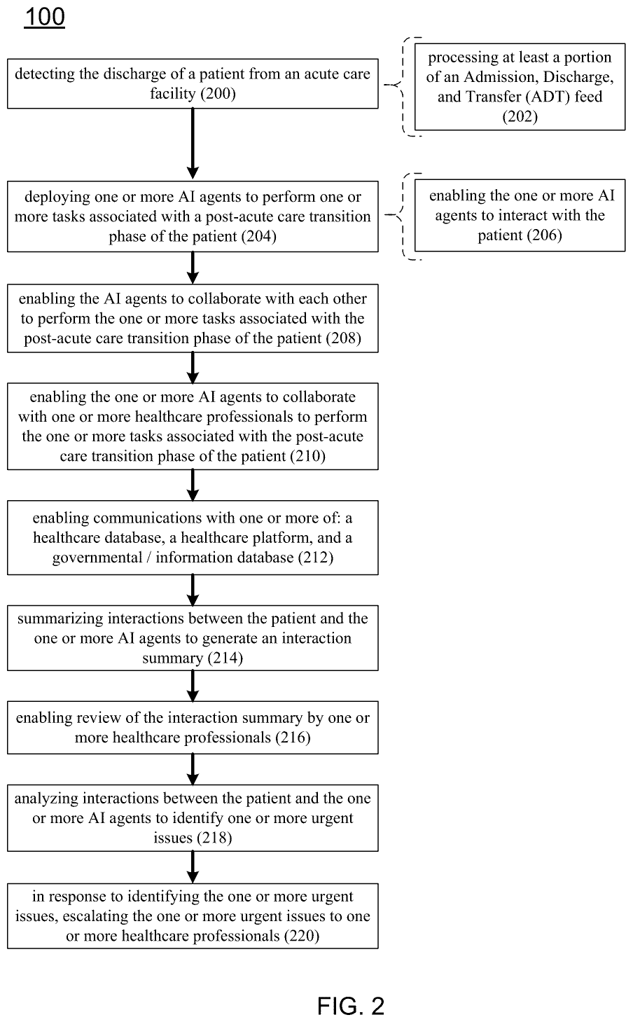

Referring to FIGS. 1-2, post-acute care transition process 100 may detect 200 the discharge of a patient (e.g., patient 102) from an acute care facility (e.g., acute care facility 104).

An acute care facility (e.g., acute care facility 104) is a healthcare institution that provides short-term treatment for patients experiencing severe injuries, illnesses, or medical conditions requiring immediate and intensive medical attention. These facilities are designed to handle urgent and critical health situations and are typically staffed with healthcare professionals, including doctors, nurses, and specialists, who are trained to provide rapid and comprehensive care.

These facilities may offer a wide range of specialized services, including emergency care, intensive care units (ICUs), surgical services, diagnostic imaging, and laboratory testing. Care may be provided by a multidisciplinary team of healthcare professionals, including emergency physicians, surgeons, internists, nurses, and respiratory therapists, to ensure comprehensive and coordinated care. Acute care facilities may be part of larger hospitals or standalone emergency departments, equipped with the necessary infrastructure, technology, and medical supplies to handle a variety of acute medical situations.

The purpose of acute care facilities (e.g., acute care facility 104) is to save lives, prevent complications, and provide immediate relief from severe health conditions. These facilities often play a crucial role in the healthcare system by ensuring that patients with acute medical needs receive timely and effective treatment, thereby reducing the risk of mortality and serious health outcomes. Once the acute phase is managed, patients may be transitioned to other levels of care, such as post-acute care, for continued recovery and rehabilitation.

When detecting 200 the discharge of a patient (e.g., patient 102) from an acute care facility (e.g., acute care facility 104), post-acute care transition process 100 may process 202 at least a portion of an Admission, Discharge, and Transfer (ADT) feed (e.g., ADT feed 106).

An Admission, Discharge, and Transfer (ADT) feed (e.g., ADT feed 106) is a component in healthcare information systems that electronically communicates patient administrative events such as admissions, discharges, and transfers. This feed (e.g., ADT feed 106) ensures real-time updates of patient information across various systems and departments, enabling seamless coordination and management of patient care.

When a patient (e.g., patient 102) is admitted, the ADT feed (e.g., ADT feed 106) may capture and transmit essential details like demographics, reason for admission, assigned bed, and attending physician, updating the hospital's electronic health record (EHR) system. This synchronization may aid in planning and resource allocation, ensuring that necessary medical supplies and medications are available.

Upon discharge, the ADT feed (e.g., ADT feed 106) may record and communicate details such as discharge date, diagnosis, destination, and follow-up care instructions. This may facilitate coordination among departments responsible for discharge planning, transportation, and home care services, preventing readmissions and ensuring continuous support.

For patient transfers, whether within the same facility or to a different facility, the ADT feed (e.g., ADT feed 106) may track and relay information about the transfer location, time, and updated care team. This may maintain continuity of care and keep all involved parties informed of the patient's status.

The real-time updates provided by ADT feeds (e.g., ADT feed 106) may be vital for maintaining accurate patient records, improving care coordination, and enhancing communication between healthcare systems. Automated ADT feeds (e.g., ADT feed 106) may reduce manual data entry, minimize administrative errors and ensure consistent information access. In Health Information Exchanges (HIEs), ADT feeds (e.g., ADT feed 106) may support continuity of care when patients move between providers. In clinical decision support systems (CDSS), ADT feeds (e.g., ADT feed 106) may offer timely data for recommendations and alerts to clinicians. Additionally, ADT feeds (e.g., ADT feed 106) may contribute to population health management by tracking patient outcomes and movements, aiding analytics and reporting.

In summary, ADT feeds (e.g., ADT feed 106) may be essential for ensuring real-time, accurate communication of patient administrative events within and across healthcare facilities. ADT feeds (e.g., ADT feed 106) may support efficient and coordinated patient care, improve operational efficiency, enhance patient safety, and facilitate better health outcomes.

Post-acute care transition process 100 may deploy 204 one or more AI agents (e.g., AI agents 108) to perform one or more tasks (e.g., tasks 110) associated with a post-acute care transition phase of the patient (e.g., patient 102).

An artificial intelligence (AI) agent (e.g., AI agents 108), generally speaking, is a computer program or system designed to perceive its environment, reason about the information it receives, and take actions to achieve specific goals or tasks autonomously. AI agents (e.g., AI agents 108) may leverage various techniques and algorithms to simulate intelligent behavior, including machine learning, natural language processing, expert systems, and symbolic reasoning.

AI agents (e.g., AI agents 108) may be categorized based on their level of autonomy and complexity:

Reactive Agents: These agents may operate based on preprogrammed rules and may not maintain an internal state or memory of past interactions. They may react directly to input stimuli without considering the broader context or history. Examples may include basic game-playing AI or systems that perform specific tasks based on fixed rules.

Limited Memory Agents: These agents may have a limited memory or ability to retain past experiences and use this information to inform their decisions. They may adapt their behavior based on recent events or interactions but may not have a comprehensive understanding of long-term patterns or dynamics in their environment.

Utility-based Agents: These agents may evaluate different actions based on their expected utility or value in achieving a particular goal. They may weigh various factors and potential outcomes to select the most advantageous course of action. Utility-based agents may be commonly used in decision-making tasks where multiple competing objectives need to be considered.

Learning Agents: These agents may improve their performance over time by learning from experience or training data. They may use techniques such as reinforcement learning, supervised learning, or unsupervised learning to update their behavior based on feedback from their environment. Learning agents may adapt to changing conditions and optimize their strategies to achieve better outcomes over time.

AI agents (e.g., AI agents 108) may be found in various applications and domains, including robotics, autonomous vehicles, virtual assistants, gaming, healthcare, finance, and more. They may enable automation, enhance decision-making capabilities, and unlock new possibilities for solving complex problems across a wide range of industries and contexts.

AI agents (e.g., AI agents 108) that perform tasks associated with the post-acute care transition phase of a patient may play a crucial role in ensuring smooth and effective continuity of care. These agents (e.g., AI agents 108) may automate a variety of tasks to support patients as they move from acute care facilities to post-acute settings, such as home care, rehabilitation centers, or long-term care facilities. By leveraging advanced technologies, these AI agents (e.g., AI agents 108) may operate in an automated, multi-modal manner, including through text, email, chatbots, and other communication channels.

Examples of such AI agents (e.g., AI agents 108) may include but are not limited to one or more of: a transitions of care history interview and discharge review agent; a medication reconciliation agent; a medical knowledge query response agent; a community resources agent; and an appointment scheduling and confirmation agent.

Transitions of Care History Interview and Discharge Review Agent: In the context of post-acute care transitions, a Transitions of Care History Interview and Discharge Review Agent may be designed to automate and enhance the process of collecting comprehensive patient histories and reviewing discharge plans. This AI agent may conduct virtual interviews with patients (e.g., patient 102) using natural language processing (NLP) to gather detailed medical histories, current health statuses, and any other relevant information. This AI agent may review discharge instructions and ensures patients understand their care plans, medications, and follow-up requirements. By leveraging AI, this agent may identify potential issues, provide personalized recommendations, and alert healthcare providers to any concerns that need attention, thereby ensuring a smoother transition and reducing the risk of readmissions.

Medication Reconciliation Agent: A Medication Reconciliation Agent in post-acute care transitions may automate the process of compiling and verifying a patient's medication list. This AI agent may use data from electronic health records (EHRs), pharmacy databases, and patient self-reports to create a comprehensive list of all medications the patient (e.g., patient 102) is taking. This AI agent may compare this list with new medication orders to identify discrepancies, potential drug interactions, and duplications. The AI agent may provide real-time alerts to healthcare providers and patients about necessary adjustments, ensuring medication accuracy and safety throughout the transition phase.

Medical Knowledge Query Response Agent: In the post-acute care transition phase, a Medical Knowledge Query Response Agent may act as a sophisticated tool for healthcare providers and patients seeking information on medical conditions, treatments, and care protocols. This AI agent may use advanced algorithms to search medical databases, clinical guidelines, and evidence-based resources to provide accurate and relevant answers to queries. This AI agent may support healthcare providers by offering quick access to medical knowledge, thus aiding in clinical decision-making and ensuring that patients receive informed and up-to-date care instructions during their transition.

Community Resources Agent: A Community Resources Agent in the context of post-acute care transitions may connect patients and their families with local community services that can support their ongoing health and well-being. This AI agent may use data on available community resources such as social services, support groups, transportation services, meal programs, and housing assistance. This AI agent may assess the specific needs of patients based on their medical history, social determinants of health, and personal preferences, and may then provide tailored recommendations. By facilitating access to these resources, the AI agent may help to address non-medical factors that can influence recovery and quality of life during the transition phase.

Appointment Scheduling and Confirmation Agent: An Appointment Scheduling and Confirmation Agent may streamline the process of booking and managing follow-up appointments for patients transitioning from acute to post-acute care. This AI agent may use machine learning algorithms to coordinate with healthcare providers' schedules and find suitable appointment times. This AI agent may send automated reminders and confirmations to patients via text, email, or phone calls, reducing the likelihood of missed appointments. The agent may also handle rescheduling requests and cancellations, ensuring that patients maintain necessary follow-up care and adhere to their post-acute care plans, thereby supporting better health outcomes and continuity of care.

By integrating these AI agents (e.g., AI agents 108) into the post-acute care transition phase, healthcare providers may enhance efficiency, improve patient outcomes, and ensure a smoother and more coordinated transition process for patients (e.g., patient 102) moving from acute to post-acute care settings.

When deploying 204 one or more AI agents (e.g., AI agents 108) to perform one or more tasks (e.g., tasks 110) associated with a post-acute care transition phase of the patient (e.g., patient 102), post-acute care transition process 100 may enable 206 the one or more AI agents (e.g., AI agents 108) to interact with the patient (e.g., patient 102).

Having AI agents (e.g., AI agents 108) interact with a patient (e.g., patient 102) to perform post-acute care transition tasks (e.g., tasks 110) may mean utilizing artificial intelligence to directly engage with patients (e.g., patient 102) as they transition from hospital (e.g., acute care facility 104) to home or another care setting. This interaction typically involves AI-driven systems such as virtual assistants or chatbots that communicate with patients (e.g., patient 102) to provide essential information, guidance, and support. These AI agents (e.g., AI agents 108) can perform a variety of tasks (e.g., tasks 110), including reminding patients about medication schedules, providing instructions for wound care or rehabilitation exercises, scheduling follow-up appointments with healthcare providers, and answering questions about their care plan. These AI agents (e.g., AI agents 108) can also monitor the patient's progress and symptoms through feedback and data collected from wearable health devices, using this information to adjust care plans or alert medical professionals if issues arise. This direct interaction aims to enhance patient engagement and adherence to prescribed care regimens, reduce the risk of complications, and improve overall outcomes by offering continuous support and personalized attention throughout the post-acute care period.

Post-acute care transition process 100 may enable 208 the AI agents (e.g., AI agents 108) to collaborate with each other to perform the one or more tasks (e.g., tasks 110) associated with the post-acute care transition phase of the patient (e.g., patient 102).

Having AI agents (e.g., AI agents 108) collaborate to perform post-acute care transition tasks (e.g., tasks 110) means employing multiple artificial intelligence systems that work together to ensure smooth transitions for patients (e.g., patient 102) moving from hospital care to home or another care setting. This collaboration may involve various specialized AI agents that manage different aspects of a patient's care. For instance, one AI agent may handle the collection and analysis of medical data from hospital records and wearable devices, providing insights into a patient's health status and recovery progress. Another AI agent may focus on scheduling follow-up appointments and coordinating with primary care providers and specialists to ensure continuity of care. Additionally, an AI agent may be dedicated to medication management, sending reminders to patients about dosages and monitoring adherence to prevent complications. These AI agents may need to communicate and share data effectively to create a seamless care experience, which may help reduce the risk of readmissions and improve overall patient outcomes by ensuring that every aspect of the transition is covered comprehensively and efficiently.

Post-acute care transition process 100 may enable 210 the one or more AI agents (e.g., AI agents 108) to collaborate with one or more healthcare professionals (e.g., healthcare professionals 112) to perform the one or more tasks (e.g., tasks 110) associated with the post-acute care transition phase of the patient (e.g., patient 102).

Having AI agents (e.g., AI agents 108) collaborate with one or more medical professionals (e.g., healthcare professionals 112) to perform post-acute care transition tasks (e.g., tasks 110) may involve integrating artificial intelligence tools with human expertise to enhance the management and continuity of care as patients move from hospital settings to home care or other secondary care facilities. This collaboration may leverage AI's capacity for handling large datasets and performing complex analyses quickly, allowing for real-time monitoring, predictive analytics, and personalized patient management strategies. For example, an AI system might analyze a patient's medical history and ongoing health data to predict potential complications, which may then be communicated to healthcare professionals (e.g., healthcare professionals 112) for timely intervention. Meanwhile, other AI functionalities may include scheduling follow-up appointments, reminding patients about medication schedules, or even instructing patients on self-care procedures through interactive platforms. These AI-driven tasks may be coordinated with the direct clinical inputs and oversight from healthcare professionals (e.g., healthcare professionals 112), ensuring that the care delivered is both technically accurate and appropriately tailored to individual patient needs. This synergistic approach aims to streamline workflows, reduce the burden on medical staff, and ultimately enhance patient outcomes by providing a more cohesive and responsive care transition experience.

Post-acute care transition process 100 may enable 212 communications with one or more of: a healthcare database, a healthcare platform, and a governmental/information database (e.g., collectively databases/platforms 214).

AI agents (e.g., AI agents 108) accessing healthcare databases, healthcare platforms, and governmental or informational databases (e.g., collectively databases/platforms 214) may significantly enhance the scope and quality of healthcare delivery by integrating diverse data sources for a more nuanced approach to patient care and health management.

Comprehensive Patient Profiles: Through access to healthcare databases, AI agents (e.g., AI agents 108) may pull together detailed and multifaceted patient profiles. These profiles may encompass not just medical histories and current health conditions, but also genetic information, past treatments, and responses to those treatments. This comprehensive data collection may allow AI agents (e.g., AI agents 108) to perform sophisticated analyses to tailor treatment plans that are uniquely suited to each patient's specific circumstances. This may enable a precision medicine approach where treatments may be individualized based on a deep understanding of a patient's medical history and personal health trajectory.

Real-Time Monitoring and Intervention: Healthcare platforms may offer real-time data feeds from clinical settings and patient monitoring devices. AI agents (e.g., AI agents 108) may utilize this real-time data to track patient health moment-by-moment, providing opportunities for timely interventions before acute conditions worsen. For instance, AI-driven systems may alert healthcare professionals (e.g., healthcare professionals 112) if a patient's vitals indicate a potential heart failure or if blood sugar levels fall outside safe parameters in diabetic patients, enabling swift action to prevent severe complications.

Regulatory Adherence and Updated Care Standards: Access to governmental databases may ensures that AI agents (e.g., AI agents 108) are constantly updated with the latest healthcare regulations and compliance requirements. This may be critical in maintaining the legality and safety of medical practices. Additionally, these databases may provide updates on clinical guidelines and standards of care, ensuring that the AI-driven decisions are based on the most current medical research and consensus in the field.

Social and Environmental Insights: Governmental and informational databases may also provide insights into social determinants of health, such as community health statistics, pollution levels, race statistics, gender statistics, and access to medical services. AI agents (e.g., AI agents 108) may use this data to understand how external factors may impact individual health outcomes. For example, an AI system may adjust a health management plan for a patient with respiratory issues by considering air quality data from their living environment.

Epidemiological Insights for Public Health Decisions: On a broader scale, access to public health data may allow AI agents (e.g., AI agents 108) to analyze trends and patterns that affect entire populations. This capability may be particularly important in anticipating health crises, such as predicting outbreaks of infectious diseases or identifying the need for health interventions in communities showing rising trends in chronic diseases.

By leveraging these diverse databases, AI agents (e.g., AI agents 108) may offer a more informed, responsive, and proactive healthcare system that not only caters to the individual needs of patients but also addresses larger public health challenges. This results in a healthcare delivery model that is not only reactive to illnesses but also proactive in maintaining health and preventing disease, guided by a deep and constantly updated pool of data.

Post-acute care transition process 100 may summarize 214 interactions between the patient (e.g., patient 102) and the one or more AI agents (e.g., AI agents 108) to generate an interaction summary (e.g., interaction summary 116).

Summarizing 214 interactions between the patient (e.g., patient 102) and the AI agents (e.g., AI agents 108) may involve condensing the exchanges and activities that occur between the patient (e.g., patient 102) and the AI agents (e.g., AI agents 108) into a concise overview (e.g., interaction summary 116). This summary (e.g., interaction summary 116) may capture the key points of the interaction, including the tasks performed, information exchanged, decisions made, and outcomes achieved.

The interaction summary (e.g., interaction summary 116) may aim to distill complex interactions into easily digestible information that can be quickly reviewed by healthcare professionals (e.g., healthcare professionals 112) or other stakeholders. The interaction summary (e.g., interaction summary 116) may include details such as the purpose of the interaction (e.g., medication reminder, symptom monitoring), any notable observations or changes in the patient's condition, actions taken by the AI agent (e.g., AI agents 108) in response to the patient's input, and any follow-up recommendations or next steps.

Post-acute care transition process 100 may enable 216 review of the interaction summary (e.g., interaction summary 116) by one or more healthcare professionals (e.g., healthcare professionals 112). Enabling the review of interaction summaries (e.g., interaction summary 116) by one or more healthcare professionals (e.g., healthcare professionals 112) may mean providing access to condensed overviews of patient interactions with AI agents (e.g., AI agents 108) for evaluation, analysis, and decision-making purposes.

By summarizing 214 interactions and enabling 216 review, healthcare professionals (e.g., healthcare professionals 112) may gain valuable insights into the patient's progress, adherence to treatment plans, and overall engagement with the AI-driven care management system. This summary (e.g., interaction summary 116) may help inform decision-making, identify areas for improvement in patient care, and facilitate communication and collaboration among members of the healthcare team. Additionally, this summary (e.g., interaction summary 116) may serve as a valuable record of the patient's interactions with the AI agents (e.g., AI agents 108), providing a comprehensive overview of their care journey over time.

Examples of such a process may include:

Access to Summaries: Healthcare professionals (e.g., healthcare professionals 112), such as doctors, nurses, or care coordinators, may be granted access to the interaction summaries (e.g., interaction summary 116) generated by AI agents (e.g., AI agents 108). These summaries (e.g., interaction summary 116) may be stored in a digital platform or electronic health record system accessible to authorized personnel.

Review and Analysis: Healthcare professionals (e.g., healthcare professionals 112) may review the interaction summaries (e.g., interaction summary 116) to gain insights into the patient's interactions with AI agents (e.g., AI agents 108). They may analyze the summaries (e.g., interaction summary 116) to understand the nature of the interactions, assess the effectiveness of the AI-driven interventions, and evaluate the patient's response to the care management strategies employed.

Decision-Making Support: The information gleaned from the interaction summaries (e.g., interaction summary 116) may inform decision-making regarding the patient's care plan. Healthcare professionals (e.g., healthcare professionals 112) may use the insights gained to make adjustments to medication regimens, treatment plans, or follow-up care protocols. They may also identify areas where additional support or intervention is needed and devise strategies to address any gaps or challenges observed.

Collaboration and Communication: Interaction summaries (e.g., interaction summary 116) may facilitate collaboration and communication among members of the healthcare team. Healthcare professionals (e.g., healthcare professionals 112) may share insights, observations, and recommendations based on the interaction summaries, enabling multidisciplinary teams to coordinate care effectively and ensure continuity of care for the patient.

Documentation and Record-Keeping: Interaction summaries (e.g., interaction summary 116) may serve as documentation of the patient's interactions with AI agents (e.g., AI agents 108), providing a comprehensive record of their care journey. Healthcare professionals (e.g., healthcare professionals 112) can refer back to these summaries (e.g., interaction summary 116) over time to track changes in the patient's condition, monitor progress, and assess the effectiveness of interventions over time.

Overall, enabling 216 the review of interaction summaries (e.g., interaction summary 116) by healthcare professionals (e.g., healthcare professionals 112) may enhance the quality-of-care delivery by providing valuable insights, supporting informed decision-making, fostering collaboration among care team members, and maintaining comprehensive records of patient interactions and outcomes.

Post-acute care transition process 100 may analyze 218 interactions between the patient (e.g., patient 102) and the one or more AI agents (e.g., AI agents 108) to identify one or more urgent issues (e.g., urgent issues 118). In response to identifying the one or more urgent issues (e.g., urgent issues 118), post-acute care transition process 100 may escalate 220 the one or more urgent issues (e.g., urgent issues 118) to one or more healthcare professionals (e.g., healthcare professionals 112).

Post-acute care transition process 100 may use AI to analyze interactions between a patient (e.g., patient 102) and the one or more AI agents (e.g., AI agents 108) to identify urgent issues (e.g., urgent issues 118) through several advanced techniques and processes.

Natural Language Processing (NLP) algorithms may analyze text or speech data from interactions (e.g., such as those defined within interaction summary 116), interpreting human language to identify keywords, phrases, or patterns that indicate potential urgent issues (e.g., urgent issues 118), such as mentions of severe pain or distress. Sentiment analysis may evaluate the emotional tone of the patient's communications, detecting negative emotions like anxiety or frustration, which may signal underlying urgent issues. Machine learning models may recognize patterns in patient behavior and responses that typically precede urgent issues, such as a sudden increase in health-related complaints or deviations from normal interaction patterns. Clinical rule-based systems may use predefined clinical rules and thresholds based on medical guidelines to flag urgent symptoms or measurements, like extremely high blood pressure or severe chest pain. Anomaly detection methods may identify deviations from the patient's baseline health status, while integration with wearable health monitors and other medical devices may track vital signs in real-time, triggering alerts for critical readings.

Post-acute care transition process 100 may consider the context of interactions by combining multiple data sources, such as recent medical history, medication changes, and known health conditions, to make more informed assessments of urgency. Real-time processing capabilities may enable immediate analysis and identification of urgent issues (e.g., urgent issues 118) as they occur, ensuring timely alerts. Post-acute care transition process 100 may automatically generate detailed alerts and notifications to healthcare professionals (e.g., healthcare professionals 112), including information about the issue, the analysis that led to its identification, and any immediate actions taken by the AI agents (e.g., AI agents 108). Continuous learning may allow post-acute care transition process 100 to improve over time by learning from past interactions and the outcomes of escalated issues, retraining machine learning algorithms with new data to enhance accuracy in identifying urgent issues (e.g., urgent issues 118). By leveraging these techniques, post-acute care transition process 100 may effectively monitor and analyze patient interactions, promptly identifies urgent issues (e.g., urgent issues 118), and ensure that such issues (e.g., urgent issues 118) are escalated to healthcare professionals (e.g., healthcare professionals 112) for timely intervention, enhancing patient safety and supporting proactive healthcare management.

### System Overview

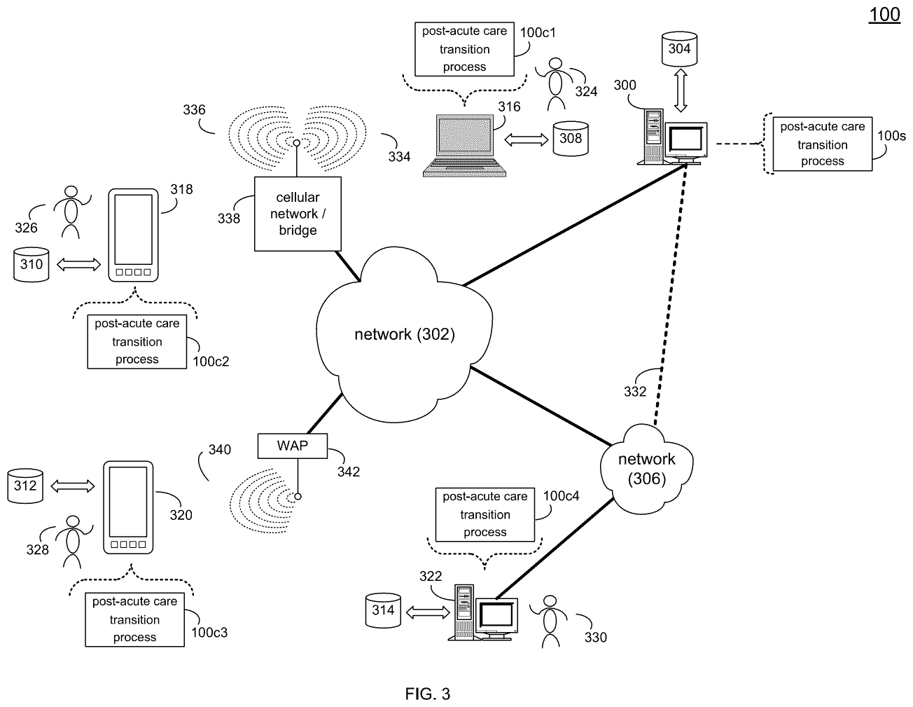

Referring to FIG. 3, there is shown post-acute care transition process 100. Post-acute care transition process 100 may be implemented as a server-side process, a client-side process, or a hybrid server-side/client-side process. For example, post-acute care transition process 100 may be implemented as a purely server-side process via post-acute care transition process 100s. Alternatively, post-acute care transition process 100 may be implemented as a purely client-side process via one or more of post-acute care transition process 100cl, post-acute care transition process 100c2, post-acute care transition process 100c3, and post-acute care transition process 100c4. Alternatively still, post-acute care transition process 100 may be implemented as a hybrid server-side/client-side process via post-acute care transition process 100s in combination with one or more of post-acute care transition process 100cl, post-acute care transition process 100c2, post-acute care transition process 100c3, and post-acute care transition process 100c4.

Accordingly, post-acute care transition process 100 as used in this disclosure may include any combination of post-acute care transition process 100s, post-acute care transition process 100cl, post-acute care transition process 100c2, post-acute care transition process 100c3, and post-acute care transition process 100c4.

Post-acute care transition process 100s may be a server application and may reside on and may be executed by computing device 300, which may be connected to network 302 (e.g., the Internet or a local area network). Examples of computing device 300 may include, but are not limited to: a personal computer, a server computer, a series of server computers, a mini computer, a mainframe computer, a smartphone, or a cloud-based computing platform.

The instruction sets and subroutines of post-acute care transition process 100s, which may be stored on storage device 304 coupled to computing device 300, may be executed by one or more processors (not shown) and one or more memory architectures (not shown) included within computing device 300. Examples of storage device 304 may include but are not limited to: a hard disk drive; a RAID device; a random-access memory (RAM); a read-only memory (ROM); and all forms of flash memory storage devices.

Network 302 may be connected to one or more secondary networks (e.g., network 306), examples of which may include but are not limited to: a local area network; a wide area network; or an intranet, for example.

Examples of post-acute care transition processes 300c1, 300c2, 300c3, 300c4 may include but are not limited to a web browser, a game console user interface, a mobile device user interface, or a specialized application (e.g., an application running on e.g., the Android™ platform, the iOS™ platform, the Windows™ platform, the Linux™ platform or the UNIX™ platform). The instruction sets and subroutines of post-acute care transition processes 300c1, 300c2, 300c3, 300c4, which may be stored on storage devices 308, 310, 312, 314 (respectively) coupled to client electronic devices 316, 318, 320, 322 (respectively), may be executed by one or more processors (not shown) and one or more memory architectures (not shown) incorporated into client electronic devices 316, 318, 320, 322 (respectively). Examples of storage devices 308, 310, 312, 314 may include but are not limited to: hard disk drives; RAID devices; random access memories (RAM); read-only memories (ROM), and all forms of flash memory storage devices.

Examples of client electronic devices 316, 318, 320, 322 may include, but are not limited to a personal digital assistant (not shown), a tablet computer (not shown), laptop computer 316, smart phone 318, smart phone 320, personal computer 322, a notebook computer (not shown), a server computer (not shown), a gaming console (not shown), and a dedicated network device (not shown). Client electronic devices 316, 318, 320, 322 may each execute an operating system, examples of which may include but are not limited to Microsoft Windows™, Android™, iOS™, Linux™, or a custom operating system.

Users 324, 326, 328, 330 may access post-acute care transition process 10 directly through network 302 or through secondary network 306. Further, post-acute care transition process 10 may be connected to network 302 through secondary network 306, as illustrated with link line 332.

The various client electronic devices (e.g., client electronic devices 316, 318, 320, 322) may be directly or indirectly coupled to network 302 (or network 306). For example, laptop computer 316 and smart phone 318 are shown wirelessly coupled to network 302 via wireless communication channels 334, 336 (respectively) established between laptop computer 316, smart phone 318 (respectively) and cellular network/bridge 338, which is shown directly coupled to network 302.

Further, smart phone 320 is shown wirelessly coupled to network 302 via wireless communication channel 340 established between smart phone 320 and wireless access point (i.e., WAP) 342, which is shown directly coupled to network 302. Additionally, personal computer 322 is shown directly coupled to network 306 via a hardwired network connection.

WAP 342 may be, for example, an IEEE 802.11a, 802.11b, 802.11g, 802.11n, Wi-Fi, and/or Bluetooth device that is capable of establishing wireless communication channel 340 between smart phone 320 and WAP 342. As is known in the art, IEEE 802.11x specifications may use Ethernet protocol and carrier sense multiple access with collision avoidance (i.e., CSMA/CA) for path sharing. As is known in the art, Bluetooth is a telecommunications industry specification that allows e.g., mobile phones, computers, and personal digital assistants to be interconnected using a short-range wireless connection.

### General:

As will be appreciated by one skilled in the art, the present disclosure may be embodied as a method, a system, or a computer program product. Accordingly, the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present disclosure may take the form of a computer program product on a computer-usable storage medium having computer-usable program code embodied in the medium.

Any suitable computer usable or computer readable medium may be used. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. The computer-usable or computer-readable medium may also be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, RF, etc.

Computer program code for carrying out operations of the present disclosure may be written in an object-oriented programming language. However, the computer program code for carrying out operations of the present disclosure may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network/a wide area network/the Internet.

The present disclosure is described with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, may be implemented by computer program instructions. These computer program instructions may be provided to a processor of a general-purpose computer/special purpose computer/other programmable data processing apparatus, such that the instructions, which execute via the processor of the computer or other programmable data processing apparatus, create means for implementing the functions/acts specified in the flowchart and/or block diagram block or blocks.

These computer program instructions may also be stored in a computer-readable memory that may direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The flowcharts and block diagrams in the figures may illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, not at all, or in any combination with any other flowcharts depending upon the functionality involved. It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

A number of implementations have been described. Having thus described the disclosure of the present application in detail and by reference to embodiments thereof, it will be apparent that modifications and variations are possible without departing from the scope of the disclosure defined in the appended claims.

What is claimed is:

1. A computer-implemented method, executed on a computing device, comprising:
detecting the discharge of a patient from an acute care facility;
deploying one or more AI agents to perform one or more tasks associated with a post-acute care transition phase of the patient; and
analyzing interactions between the patient and the one or more AI agents to identify one or more urgent issues.

2. The computer-implemented method of claim 1 wherein detecting the discharge of a patient from an acute care facility includes:
processing at least a portion of an Admission, Discharge, and Transfer (ADT) feed.

3. The computer-implemented method of claim 1 wherein the one or more AI agents includes one or more of the following:
a transitions of care history interview and discharge review agent;
a medication reconciliation agent;
a medical knowledge query response agent;
a community resources agent; and
an appointment scheduling and confirmation agent.

4. The computer-implemented method of claim 1 further comprising:
in response to identifying the one or more urgent issues, escalating the one or more urgent issues to one or more healthcare professionals.

5. The computer-implemented method of claim 1 wherein deploying one or more AI agents to perform one or more tasks associated with a post-acute care transition phase of the patient includes:
enabling the one or more AI agents to interact with the patient.

6. The computer-implemented method of claim 5 further comprising:
summarizing interactions between the patient and the one or more AI agents to generate an interaction summary.

7. The computer-implemented method of claim 6 further comprising:
enabling review of the interaction summary by one or more healthcare professionals.

8. The computer-implemented method of claim 1 further comprising:
enabling the AI agents to collaborate with each other to perform the one or more tasks associated with the post-acute care transition phase of the patient.

9. The computer-implemented method of claim 1 further comprising:
enabling the one or more AI agents to collaborate with one or more healthcare professionals to perform the one or more tasks associated with the post-acute care transition phase of the patient.

10. The computer-implemented method of claim 1 further comprising:
enabling communications with one or more of: a healthcare database, a healthcare platform, and a governmental/information database.

11. A computer program product residing on a computer readable medium having a plurality of instructions stored thereon which, when executed by a processor, cause the processor to perform operations comprising:
detecting the discharge of a patient from an acute care facility;
deploying one or more AI agents to perform one or more tasks associated with a post-acute care transition phase of the patient; and
analyzing interactions between the patient and the one or more AI agents to identify one or more urgent issues.

12. The computer program product of claim 11 wherein detecting the discharge of a patient from an acute care facility includes:
processing at least a portion of an Admission, Discharge, and Transfer (ADT) feed.

13. The computer program product of claim 11 wherein the one or more AI agents includes one or more of the following:
a transitions of care history interview and discharge review agent;
a medication reconciliation agent;
a medical knowledge query response agent;
a community resources agent; and
an appointment scheduling and confirmation agent.

14. The computer program product of claim 11 further comprising:
in response to identifying the one or more urgent issues, escalating the one or more urgent issues to one or more healthcare professionals.

15. The computer program product of claim 11 wherein deploying one or more AI agents to perform one or more tasks associated with a post-acute care transition phase of the patient includes:
enabling the one or more AI agents to interact with the patient.

16. The computer program product of claim 15 further comprising:
summarizing interactions between the patient and the one or more AI agents to generate an interaction summary.

17. The computer program product of claim 16 further comprising:
enabling review of the interaction summary by one or more healthcare professionals.

18. The computer program product of claim 11 further comprising:
enabling the AI agents to collaborate with each other to perform the one or more tasks associated with the post-acute care transition phase of the patient.

19. The computer program product of claim 11 further comprising:
enabling the one or more AI agents to collaborate with one or more healthcare professionals to perform the one or more tasks associated with the post-acute care transition phase of the patient.

20. The computer program product of claim 11 further comprising:
enabling communications with one or more of: a healthcare database, a healthcare platform, and a governmental/information database.

21. A computing system including a processor and memory configured to perform operations comprising:
detecting the discharge of a patient from an acute care facility;
deploying one or more AI agents to perform one or more tasks associated with a post-acute care transition phase of the patient; and
analyzing interactions between the patient and the one or more AI agents to identify one or more urgent issues.

22. The computing system of claim 21 wherein detecting the discharge of a patient from an acute care facility includes:
processing at least a portion of an Admission, Discharge, and Transfer (ADT) feed.

23. The computing system of claim 21 wherein the one or more AI agents includes one or more of the following:
a transitions of care history interview and discharge review agent;
a medication reconciliation agent;
a medical knowledge query response agent;
a community resources agent; and
an appointment scheduling and confirmation agent.

24. The computing system of claim 21 further comprising:
in response to identifying the one or more urgent issues, escalating the one or more urgent issues to one or more healthcare professionals.

25. The computing system of claim 21 wherein deploying one or more AI agents to perform one or more tasks associated with a post-acute care transition phase of the patient includes:
enabling the one or more AI agents to interact with the patient.

26. The computing system of claim 25 further comprising:
summarizing interactions between the patient and the one or more AI agents to generate an interaction summary.

27. The computing system of claim 26 further comprising:
enabling review of the interaction summary by one or more healthcare professionals.

28. The computing system of claim 21 further comprising:
enabling the AI agents to collaborate with each other to perform the one or more tasks associated with the post-acute care transition phase of the patient.

29. The computing system of claim 21 further comprising:
enabling the one or more AI agents to collaborate with one or more healthcare professionals to perform the one or more tasks associated with the post-acute care transition phase of the patient.

30. The computing system of claim 21 further comprising:
enabling communications with one or more of: a healthcare database, a healthcare platform, and a governmental/information database.
